(19) United States
(12) Patent Application Publication
(10) Pub. No.: US 2009/0133702 A1
(43) Pub. Date: May 28, 2009

(54) Medical Spine Board

(76) Inventor: Mao-Kuan Chang, Taichung City (TW)

Correspondence Address: HDLS Patent & Trademark Services, P.O. BOX 220746, CHANTILLY, VA, 20153-0746, US

(21) Appl. No.: 11/944,667

(22) Filed: Nov. 26, 2007

(51) Int. Cl. A61B 19/00

(52) U.S. Cl. 128/870; 128/869

(57) ABSTRACT

A medical spine board includes a main board, a plurality of covering cloths and protective soft blocks, a head plate set with two fixed plates with adjustable interval. The main board is certainly strong and set with a soft back pad to support the human body. The covering cloths are set symmetrically in two sides of the main board with fastening layers and adhering layers to be fastened together. The covering cloths are also set with the soft protective blocks in detachable way. The head plate is set in front of the main board to support the human head. The fixed plates are set with soft pillow, fastening layer and adhering layer to fasten the human head. The medical support structure made in aforementioned structure can fasten each parts of the human body with soft and comfortable protection.

### BACKGROUND OF THE INVENTION

1. Field of the Invention

The present invention is related to a medical apparatus. More particularly, the present invention is related to a spine board that applies more comfortable and friendly treatment by adjusting the size of a head pillow depending on the patient's head, and fast fixing each body parts of the patient.

2. Descriptions of the Related Art

As known in general, there are two types of medical spine board or backboard. One is a straight board, perhaps set with a pad on the board, which offers only a little bit comfort for a painful wounded patient; the other is a piece of cloth mat, which is softer yet not sustaining enough, and insufficient in safety and protection for a fractured patient whose trauma needs to be fixed. However, whether it is a straight board or piece of cloth mat, mostly only some simple clasping taps or adhering taps is set on two sides of the board. The taps are usually set on the top, middle, and bottom portions of the board or mat, which roughly bind the chest, the abdomen and the legs of a human body, and cannot fix and protect the body completely. Once any deviation or slip occurs, the trauma easily gets worse, or the human body might easily slip off, which concerns us for the users.

In view of the failings derived from the aforementioned prior art, the inventor of the present invention was inclined to improve it, and finally succeeded in developing the present invention, which can fix and bind human body parts respectively, and reach to a more comfortable and friendly design.

### SUMMARY OF THE INVENTION

The present invention is related to a medical spine board which comprises:

a main board, which is a board with a pre-established size and proper strength whereon a back pad made of soft material is set to bear a human body;

a plurality of covering cloths, separately set in the main board at the left and right sides, wherein an inside of one covering cloth has a fastening layer, and an outside of another corresponding covering cloth has an adhering layer, thereby two covering cloths can be fastened to each other;

a plurality of protective soft blocks made of foamed sponges, set in certainly proper positions of two sides of the main board in detachable way; and

a head plate, set in one end of the main board to support the human head, wherein the head plate comprises two sides each being set with a fixed plate, the fixed plates and the head plate are combined together by a sliding furrow with a screw component therethrough, thereby the fixed plates changes an interval by loosing the screw component then moving the fixed plates. In addition, the two fixed plates are set with protective planks, and a fastening layer and a adhering layer for being fastened to each other.

The spine board composed with the aforementioned components provides an adjustable design depending on the user's body type. It also comprises a structure that can respectively fix human body parts and offers comfortable protection.

### BRIEF DESCRIPTION OF THE DRAWINGS

The drawings disclose an illustrative embodiment of the present invention which serves to exemplify the various advantages and objects hereof, and are as follows:

FIG. 1 is an exploded diagram of the perspective appearance of a preferred embodiment of the present invention;

FIG. 2 is an exploded diagram of the perspective appearance of the head plate of the present invention;

FIG. 3 is a perspective combination diagram of FIG. 1;

FIG. 4 is a combination diagram of the spine board, which shows a preferred embodiment of the present invention;

FIG. 5 is the top-view diagram of the using is FIG. 4; and

FIG. 6 is an exploded diagram of the perspective appearance of the present invention.

### DESCRIPTION OF THE PREFERRED EMBODIMENT

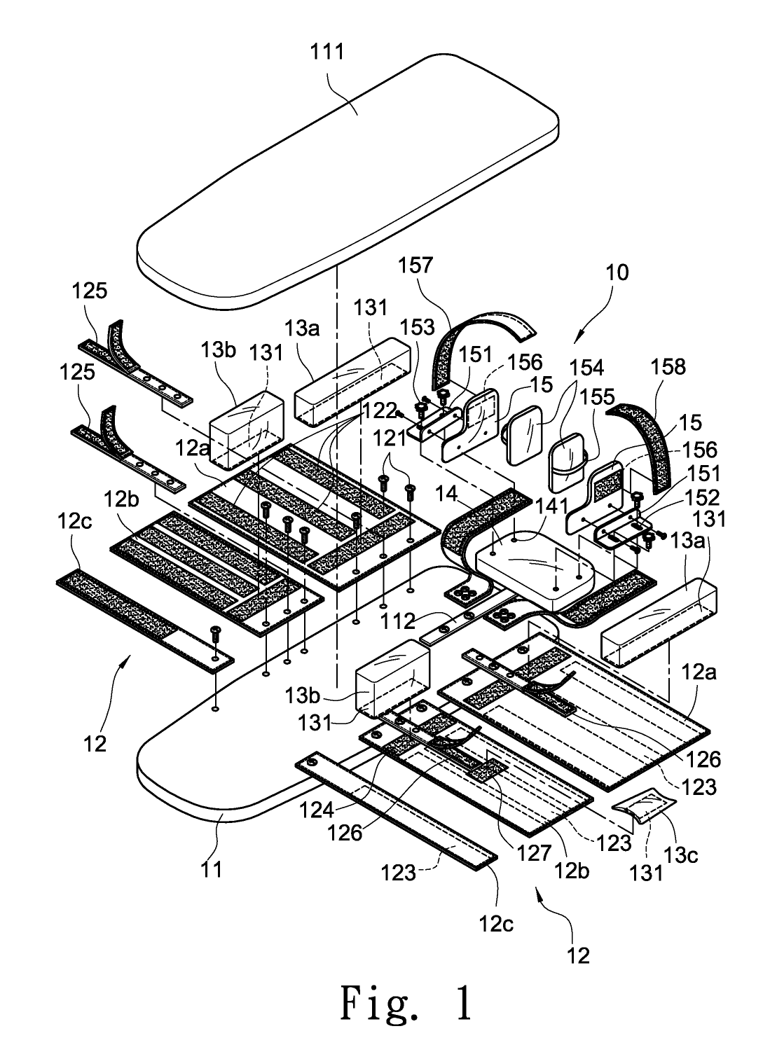

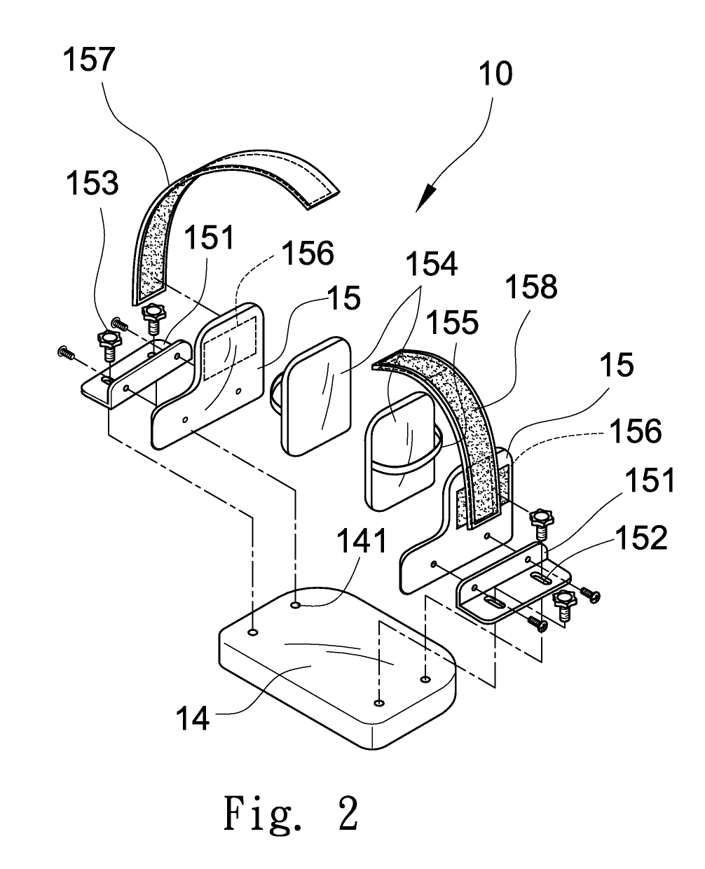

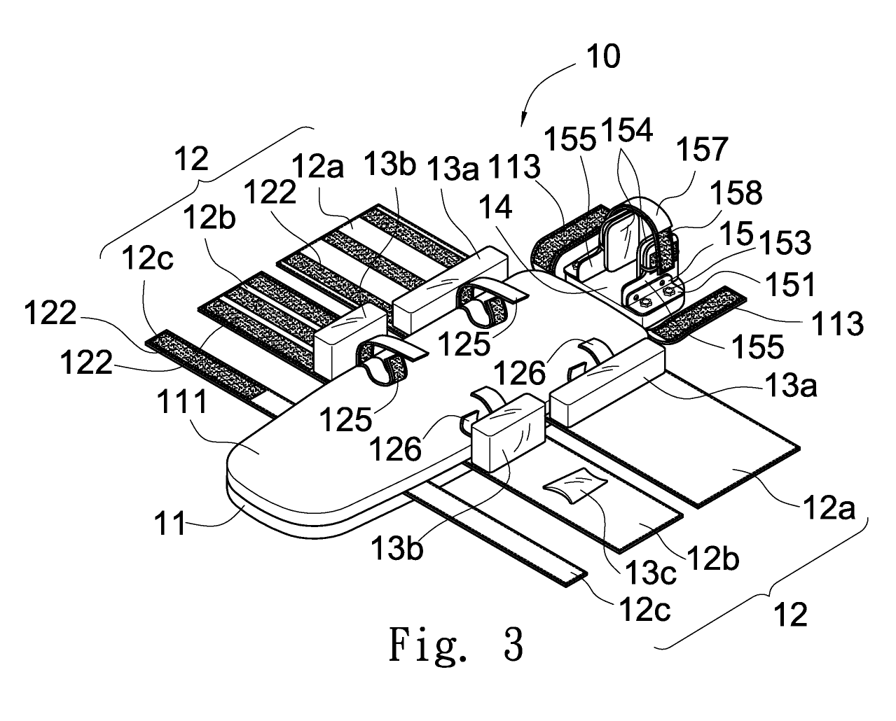

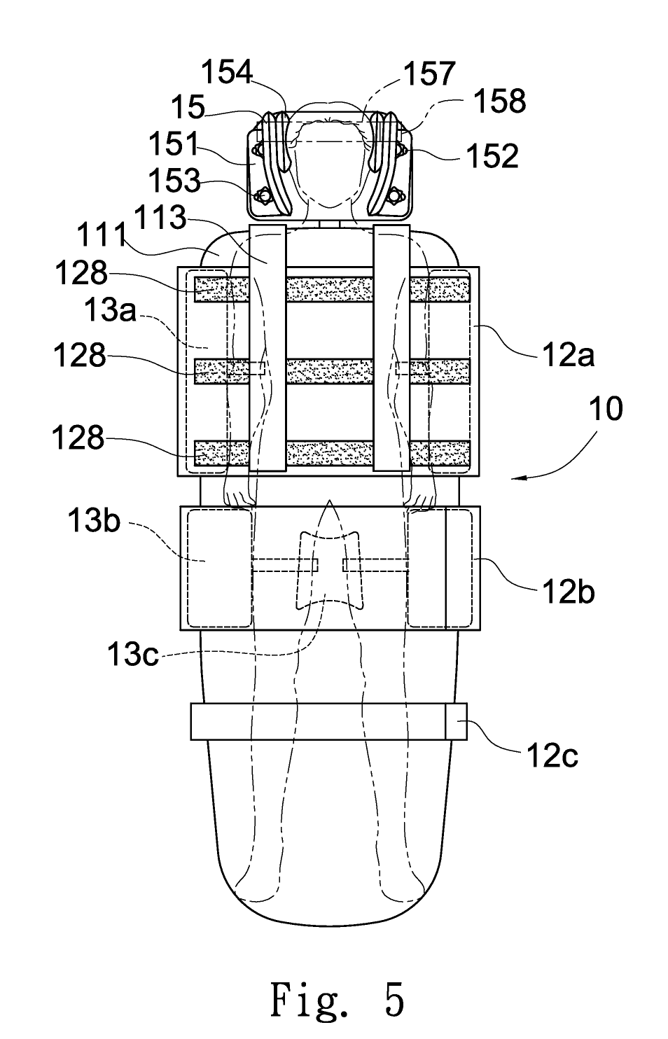

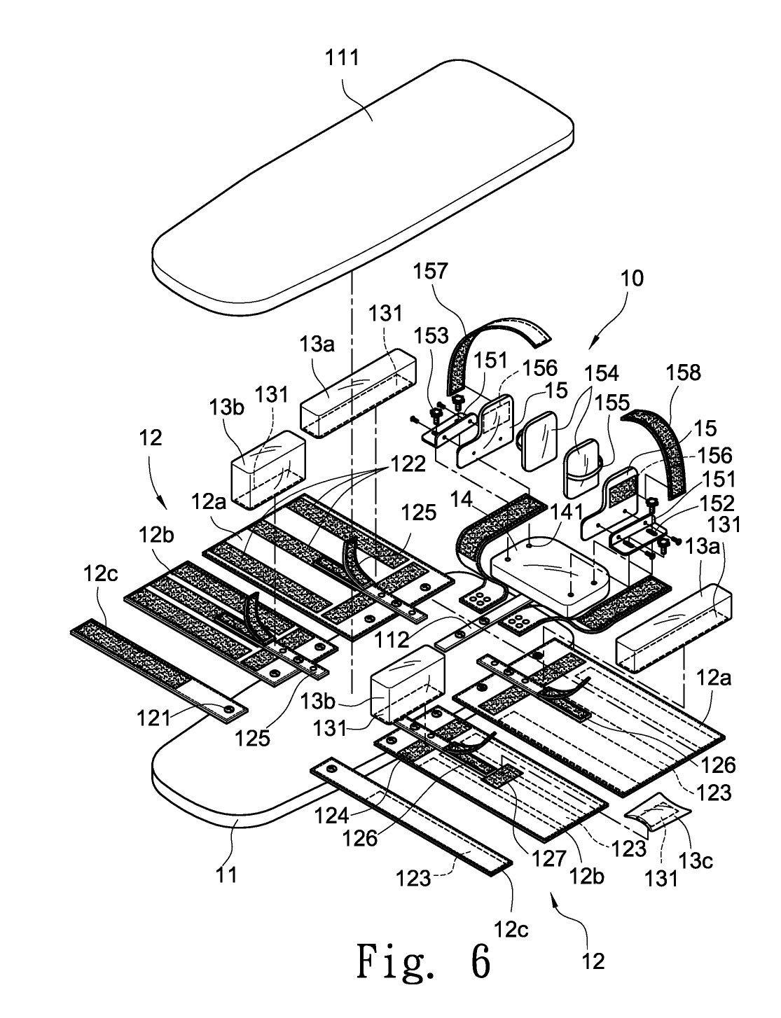

Refer to FIGS. 1-6, the present invention relates to a medical spine board 10, mainly comprising a main board 11, a plurality of covering clothes 12 and protective soft blocks 13, a head plate 14 and two fixed plates 15. The main board 11 is made of materials with a predetermined size of rectangle board and proper strength, that is, the main board 11 is configured to provide sufficient support for a human body. The main board 11 has a back pad 111 made of soft materials to provide soft and comfortable usage.

The plurality of covering clothes 12 are symmetrically set in the left and right sides of the main board 11 and screwed thereon by a plurality of screws 121. In the embodiment, the plurality of covering clothes 12 comprise a set of top covering clothes 12a, a set of middle covering clothes 12b, and a set of bottom covering clothes 12c being set symmetrically. Structural characters of the set of top covering clothes 12a, the set of middle covering clothes 12b, and the set of bottom covering clothes 12c are identical. That is, an inside of the set of covering clothes 12a, 12b, and 12c has a plurality of fastening layers 122, and an outside of another corresponding covering clothes has a plurality of adhering layers 123, thereby each two corresponding covering cloths of covering clothes 12a, 12b, and 12c being set in the left and right sides of the main board 11 can be fastened to each other. And the top covering clothes 12a can be applied to fasten the human chest and abdomen positions on the main board 11, the middle covering clothes 12b can be applied to fasten the human thigh position on the main board 11, and the bottom covering clothes 12c can be applied to fasten the human shank position on the main board 11. To fasten different positions of human body, each set of covering clothes are different in size, that is, the top covering clothes 12a are longer, and the bottom covering clothes 12c are shorter, respectively, in response to different positions of human body. More, two insides of the set of middle covering cloths 12b are set with a fastening layer 125 and an adhering layer 126 to enhance stability and protection of the human thigh.

The plurality of protective soft blocks 13 are made of foamed sponges and set in certainly proper positions of two sides of the main board 11 in detachable way. In the embodiment, a set of top protective soft blocks 13a, and a set of middle protective soft blocks 13b are designed corresponding to the length of the top covering clothes 12a and the middle covering clothes 12b. A bottom of each protective soft blocks 13a and 13b is set with an adhering layer 131, and a plurality of fastening layer 124 corresponding to the adhering layers 131 are set in each of the top covering clothes 12a and the middle covering clothes 12b. Thus the top protective soft blocks 13a and the middle protective soft blocks 13b set in the left and right sides of the top covering clothes 12a and the middle covering clothes 12b respectively are configured to buffer the human body with the protective soft blocks 13, when the top covering clothes 12a and the middle covering clothes 12b are used to fasten the human body. Therefore the human body can be free from seriously bleeding due to direct pressure applied on wounds. More, a fastening layer 127 is set in the middle position of an inside of the middle covering clothes 12b to be fastened to an adhering layer 131 of a bottom protective soft block 13c in detachable way. Thus the bottom protective soft block 13c is configured to protect a portion between the human thighs.

The head plate 14 is a square plate, and is set in front of the main board 11 by a supporting bar 112 set centrally in the main board 11 to support the human head. A top portion and a bottom portion of the left and right sides of the head plate 14 are set with screw holes 141.

The two fixed plates 15 are fixed in a frame 151 substantially in L shape. A sliding furrow 152 is set in a lateral bottom side of the frame 151, and two screw components 153 are screwed up via the screw holes 141 to fasten the fixed plates 15 and the head plate 15. Thereby the fixed plates 15 changes an interval by loosing the screw components 153 and then moving the fixed plates 15 to adjust for fastening different human heads between the two fixed plates 15. Two fixed pads 154 are made of soft materials such as foamed sponges and are directly slipped on the two fixed plates 15 by connecting belts 155 thereof. Fastening layers 156 are set in each outside of the two fixed plates 15 to be fastened to another fastening layer 157 and adhering layer 158 for connecting the two fixed plates 15 and fixing the human head on a forehead position, thus enhancing stability.

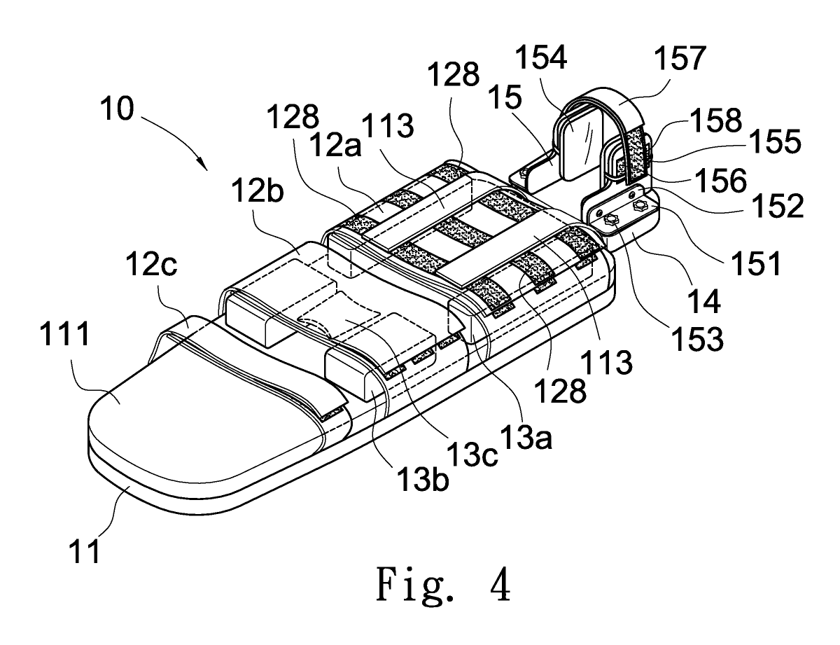

Moreover, a set of fastening layers 113 are set in two sides of the supporting bar 112 of the main board 11 and corresponding to the human shoulders position, and a set of adhering layers 128 are set in an outside of the top covering cloths 12a of the two corresponding top covering cloths 12a to be fastened to the fastening layers 113 and to provide enhanced stability, as shown in FIG. 4. Thus it can prevent the patient from slipping out of the main board 11 forwardly.

The present invention provides a medical spine board 10 as aforementioned. When a human body is put onto the main board 11, the top covering clothes 12a, the middle covering clothes 12b, and the bottom covering clothes 12c are fastened to simply and fast fix the upper half-body, thighs, and shanks. Then the fixed plates 15 are adjusted to fit the head of the patient. Then the fastening layer 157 and the adhering layer 158 are fastened to fix the head and prevent from shaking. Finally, the fastening layers 113 located in two sides of the patient's shoulders are fastened to the adhering layers 128 of the outside of the top covering clothes 12a.

The present invention is configured to certainly fix the human body with corresponding fastening structures and thus prevents the drawbacks of prior art of references that only partially cover the human body and the limbs are easy to slip over and the wounds are badly damaged. The protective soft blocks 13 are configured to protect the wounds from seriously bleeding by direct pressure that are caused by tightly fastening. Moreover, the fixed plates 15 are able to adjust the interval therebetween, and are available to fast and comfortably position kinds of people.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A spine board structure, comprising:
a main board, having a back pad made of soft materials;
a plurality of covering cloths, separately set in the main board at the left and right sides, wherein an inside of one covering cloth has a fastening layer, and an outside of another corresponding covering cloth has an adhering layer, thereby two covering cloths can be fastened to each other;
a plurality of protective soft blocks made of foamed sponges, set in certainly proper positions of two sides of the main board in detachable way;
a head plate, set in one end of the main board to support the human head, wherein the head plate comprises two sides each being set with a fixed plate, the two fixed plates are set with protective planks, and a fastening layer and a adhering layer for being fastened to each other.

2. The spine board structure as claimed in claim 1, wherein the fixed plates and the head plate are combined together by a sliding furrow with a screw component therethrough, thereby the fixed plates changes an interval by loosing the screw component then moving the fixed plates.

3. The spine board structure as claimed in claim 1, wherein a set of fastening layers are set between the main board and the head plate corresponding to the human shoulders position, and a set of adhering layers are set in an outside of one covering cloths of two corresponding covering cloths to provide enhanced stability.

4. The spine board structure as claimed in claim 1, wherein the plurality of covering cloths comprise a set of top covering cloths, a set of middle covering cloths, and a set of bottom covering cloths, and each set of covering cloths are set symmetrically in the left and the right sides of the main board.

5. The spine board structure as claimed in claim 4, wherein the set of top covering cloths are made in larger size and corresponding to the human chest position.

6. The spine board structure as claimed in claim 4, wherein the set of middle covering cloths are made in proper size and corresponding to the human thigh position.

7. The spine board structure as claimed in claim 4, wherein the set of bottom covering cloths are made in small size and corresponding to the human shank position.

8. The spine board structure as claimed in claim 4, wherein two insides of the set of middle covering cloths are set with a fastening layer and an adhering layer to enhance stability and protection of the human thigh.

9. The spine board structure as claimed in claim 4, wherein a middle position of the set of middle covering cloths is set with a bottom protective soft block to protect a portion between the human thighs.

10. The spine board structure as claimed in claim 4, wherein the protective soft blocks are attached to the top covering cloths and the middle covering cloths by additional fastening layers and adhering layers.
